SEQUENCE LISTING (1 sequence)

SEQ ID NO: 1 - DNA, 23 bp, Mycobacterium bovis
agccgtagtcgtgcagaagcgca

(19) United States
(12) Patent Application Publication
(10) Pub. No.: US 2024/0393243 A1
(43) Pub. Date: Nov. 28, 2024

(54) DETECTION OF BOVINE TUBERCULOSIS USING A GENOSENSOR (NANO-GENOSENSOR) BASED ON FLUORESCENT SEMICONDUCTOR NANOPARTICLES

(71) Applicants: María Ligia INOSTROZA CONCHA, San Pedro de la Paz (CL); UNIVERSIDAD SAN SEBASTIAN, Santiago (CL)

(72) Inventors: María Ligia INOSTROZA CONCHA, San Pedro de la Paz (CL); Patricio OYARZUN CAYO, Santiago (CL); Victor DIAZ GARCIA, Santiago (CL)

(21) Appl. No.: 18/281,180

(22) PCT Filed: Mar. 10, 2021
(86) PCT No.: PCT/CL2021/050013

§ 371 Date: Sep. 8, 2023

(51) Int. Cl. G01N 21/64; B82Y 15/00; B82Y 20/00; B82Y 40/00; C09K 11/02; C09K 11/88; C12Q 1/689

(52) U.S. Cl. CPC G01N 21/6428; C09K 11/02; C09K 11/883; C12Q 1/689; B82Y 15/00; B82Y 20/00; B82Y 40/00; C12Q 2600/156; G01N 2021/6432

(57) ABSTRACT

A method for detection of Mycobacterium bovis based on fluorescent semiconducting nanoparticle nano-genosensors (Quantum Dots, QDs), genomic probes and a quencher is provided. Upon exposure of the nano-genosensor to M. bovis DNA, the quencher is displaced, and the DNA binds to the probe bond to the fluorescent semiconductor nanoparticle composed of Cadmium Telluride (CdTe), thus causing an increase in fluorescence (turn-on). The photonic nano-genosensor emits fluorescence proportional to the amount of Mycobacterium bovis DNA present in a sample.

### TECHNICAL FIELD OF THE INVENTION

The technical field of the invention relates to the on-site detection of mycobacteria, and it can be used in the animal health sector.

### SEQUENCE LISTING

The sequence listing submitted via EFS, in compliance with 37 CFR § 1.821 (c) (1), is incorporated herein by reference. The sequence listing ASCII text file submitted via EFS contains the file “60083001USSeqST25.txt”, created on Feb. 2, 2024, which is 674 bytes in size.

### BACKGROUND OF THE INVENTION

Bovine tuberculosis is a zoonotic infectious-contagious disease caused by Mycobacterium bovis (M. bovis). It mainly affects cattle and causes chronic disease in animals and substantial economic losses associated with confiscation at slaughterhouses, reduction in animal weight, lower animal milk productivity and export restrictions.

Currently, various methods are used to detect M. bovis infection such as the skin diagnostic test known as “Tuberculin Test”, histopathological techniques, bacteriological cultures, and nucleic-acid amplification reactions (PCR). However, these methods have several disadvantages including low specificity, contamination with other microorganisms and analysis times of more than 3 days, respectively.

Among these, the Tuberculin Test is the most widely used in the field, although the technique has several drawbacks and logistical limitations associated with herd management for inoculation with a protein derivative, slow response time (72 hours post-inoculation), subjective interpretation of results by observation and palpation of the lesions, and—from a diagnostic point of view, a low specificity, since animals previously infected by environmental mycobacteria can generate false positives, and a low sensitivity derived from anergic animals, thus spreading and maintaining the disease in the herds.

On the other hand, histopathological techniques attempt to visualize the granulomatous lesion characteristic of mycobacterial infection and are generally performed in those tissues or organs that show suspicious lesions on macroscopic examination. It is a quick and relatively simple analysis, which allows a fairly accurate approximation of the infectious status of the animal in relation to this disease. However, it requires specialized personnel authorized by the health authority (Agriculture and Livestock Service) to take the sample and analyze it. It shall be mentioned that this technique is confirmatory and is performed postmortem.

On the other hand, bacteriological cultures are also a confirmatory technique in case of suspicion of bovine tuberculosis infection. The sample used in the diagnosis of bovine tuberculosis by bacteriological culture corresponds to the affected organ, which constitutes a postmortem diagnosis. In addition, the M. bovis bacterium shows difficulties in its isolation, since it requires special culture media, it grows slowly and is affected by contamination with other microorganisms.

The nucleic-acid amplification reaction—also known as the polymerase chain reaction (PCR) technique, is successfully applied for the diagnosis of bovine tuberculosis and is the only genetic amplification assay used to detect BT. This technique must be performed in highly complex laboratories and by specialized personnel preventing its use as an on-site detection technique. This reaction allows shortening the diagnostic time in relation to other techniques; although it has several drawbacks, the most common being the presence of inhibitors in the samples and frequent contamination. This limitation in the usefulness of PCR to detect M. bovis is consistent with the study of Mycobacterium tuberculosis in human medicine, as PCR is not suitable as an on-site technique, it requires highly qualified personnel and, in addition, the enzymatic reaction is particularly sensitive to the presence of inhibitors in the samples.

### STATE OF THE ART

EP227020202A1 describes a method for determining the presence of mycobacterial polynucleotides in a sample with the objective of simultaneous detection of multiple mycobacterial DNA targets without molecular amplification. The method comprises contacting the sample with one or more signal oligonucleotides and determining whether the oligonucleotides bind to mycobacterial polynucleotides in the sample, wherein at least one of the signal oligonucleotides is bound to a quantum dot, wherein the determination of such binding is optionally carried out by detecting a change in photoluminescence. It is indicated that the method enables the rapid and direct detection of the major mycobacterial pathogens [Mycobacterium tuberculosis complex (MTC), M. avium complex (MAV) and M. avium subsp. paratuberculosis (MAP)] collectively in clinical samples in a highly specific manner that is very easy to perform and requires minimal infrastructure and expertise.

In the thesis of A. Leyva (2018) entitled “Desarrollo de sondas acopladas a Quantum dots para analizar la localización subcelular del ARN genómico de VIH-1 mediante microscopia confocal” [Development of probes coupled to Quantum dots to analyze the subcellular localization of HIV-1 genomic RNA by confocal microscopy] the use of probes coupled to Cadmium-Telluride QDs is described, but specifically targeted to recognize the pBSK-GagPol region. The advantage of using QDs is having a significantly longer fluorescent lifetime, higher photostability and a broad absorption spectrum compared to the use of in-situ hybridization using organic fluorophores.

CN109655450 describes the construction and application of an electrochemical luminescence biosensor based on CdTe quantum dots aggregation and cyclic enzyme double amplification signals to improve sensitivity. In said document, water-soluble nanospheres mSQSNSs are prepared; a large number of luminophors CdTe QDs are gathered in the material, so that the material has excellent electrochemical luminescence property. Nontoxic silicon dioxide on the surface of the material greatly enhances the biocompatibility of the material; and a double-signal amplification electrochemical luminescence biosensor is constructed. A magnetic material Fe3o4-AuNPs is used as a base material, and the magnetic material has the advantages of superparamagnetism, large specific surface area, good biocompatibility, strong catalytic capacity and the like. The described biosensor is characterized by improved detection specificity and sensitivity and achieves simple and rapid detection.

ES2718084 describes fluorescent silica-based nanoparticles that enable the precise detection, characterization, monitoring and treatment of a disease such as cancer. The nanoparticle has a fluorescent compound positioned within the nanoparticle and has greater brightness and fluorescent quantum yield than the free fluorescent compound To facilitate efficient urinary excretion of the nanoparticle, it may be coated with an organic polymer, such as polyethylene glycol) (PEG) The small size of the nanoparticle, the silica base and the organic polymer coating minimizes the toxicity of the nanoparticle when administered in vivo. The nanoparticle may further be conjugated to a ligand capable of binding to a cellular component associated with the specific cell type, such as a tumor marker A therapeutic agent may be bond to the nanoparticle to permit the nanoparticle to be detectable by various imaging techniques.

Finally, application U.S. Pat. No. 9,202,867 describes nanocrystals containing CdTe core with CdS and ZnS coatings, methods for making the same, and their use in biomedical and photonic applications, such as sensors for analytes in cells and preparation of field effect transistors. In particular, the description provides a nanocrystal having a CdTe core, a CdS coating on the core, and a ZnS coating on the CdS coating, wherein the nanocrystal has a photoluminescence maximum of about 650 nm and 900 nm.

In view of this background, the development of biosensors offers the possibility of optimizing the limits of sensitivity and specificity, thus satisfying requirements of greater portability, speed and having a bioreceptor capable of selectively binding to an analyte and which in turn produces a quantifiable change during recognition. Motivated by this, in the present invention a photonic nano-genosensor was developed, which emits fluorescence proportional to the amount of M. bovis DNA present in a sample.

### BRIEF DESCRIPTION OF THE FIGURES

FIG. 1 shows a scheme of operation of the nano-genosensor. In the absence of M. bovis DNA, low fluorescence emission is present. In the presence of M. bovis DNA, high fluorescence emission is present. QBH represents the commercial quencher “Quencher Black Hole”.

FIG. 2 shows autofluorescence from different working matrices, bovine milk (white), bovine saliva (gray) and bovine milk serum (black). The wavelengths used for measurement correspond to emission in green (460±10 nm), yellow (540±10 nm) and red (590±10 nm). The fluorescence units (A.U.) range from 87485 (bovine milk) to 17 (bovine saliva).

FIG. 3 shows (A) percentage of fluorescence intensity of non-functionalized CdTe-GSH QDs and different concentrations of QBH quencher in relation to their control without quencher; (B) percentage of decrease in fluorescence intensity of functionalized CdTe-GSH QDs and different concentrations of QBH quencher in relation to their control without quencher, where GSH: glutathione.

FIG. 4 shows the calibration curve for DNA detection using a nano-genosensor. The fluorescence increase in relation to the control is shown for the nano-genosensor incubated with 0 (control), 0.125, 0.25, 0.5 and 1.0 nanomoles of M. bovis DNA. The inner image corresponds to the image of microreactors used for the detection of M. bovis DNA using the nano-genosensor of the invention.

### DESCRIPTION OF THE INVENTION

The invention provides a genosensor or nano-genosensor for on-site detection of the presence of bovine Tuberculosis, wherein the nano-genosensor comprises 3 components: fluorescent semiconductor nanoparticles (QDs) bond to genomic probes for recognition of M. bovis DNA, and a fluorescence quencher bond to a DNA sequence, complementary to the recognition probe.

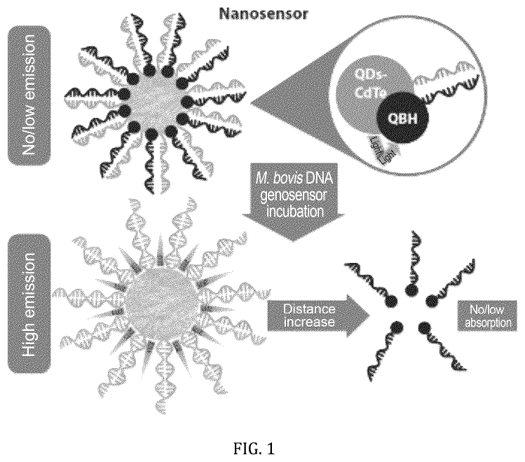

The proposed solution consists of a detection system using fluorescent semiconductor nanoparticles (QDs). This fluorescent semiconductor nanoparticle (QDs) of CdTe has a genomic probe, which is complementary to the gene to be recognized, and a quencher that is capable of absorbing the fluorescence of the nanoparticle in the absence of the gene. When the nano-genosensor is added into the matrix—with which it is desired to work, if the gene is present, the sensor separates and binds the gene to its complementary sequence, thus distancing the quencher from the fluorescent nanoparticle and obtaining an increase in fluorescence as a result. FIG. 1 shows a diagram of the operation of the nano-genosensor in the absence and presence of the analyte to be recognized.

### Fluorescent Semiconductor Nanoparticles (Quantum Dots, QDs)

In a preferred embodiment of the invention, the fluorescent semiconductor nanoparticles are composed of Cadmium Telluride (CdTe), a crystalline compound formed by the union of cadmium and tellurium having semiconductor features. The nanoparticles are made from Glutathione (GSH), cadmium chloride (CdCl₂) and potassium tellurite (K₂TeO₃).

In one embodiment of the invention, GSH, CdCl₂ and K₂TeO₃ are used in a molar ratio of 10-15:4-6:1.

### Genomic Probes

Difference region (DR)-based probes consisting of DNA segments that are present in the genome of M. bovis but have been differentially deleted from the genome of other members of the M. bovis complex were used.

In a preferred embodiment of the invention, the probe is a sequence complementary to the RD4 gene of M. bovis (SEQ. ID No. 1, AG CCG TAG TCG TGC AGA AGC GCA). The selection of the RD4 gene is based on previous studies, where a higher recognition of the RD4 gene in bovine tuberculosis positive samples is observed, being RD4 a specific gene of M. bovis.

The ratio of genomic probe to nanoparticles is 2 to 6 nmoles/mg nanoparticles.

### A Fluorescence Quencher

The quencher is a molecule that is coupled (hybridized) to the sequence complementary to the genomic probe sequence.

The fluorescence quencher is constantly absorbing the fluorescence emitted by the nanoparticle; therefore, it is possible to say that the nano-genosensor is off. Upon exposure to M. bovis DNA, the quencher is displaced, and the DNA in the sample interacts with the probe bond to the nanoparticle, causing an increase in fluorescence (turn-on) as the quencher is away from the nanoparticle.

In a preferred embodiment of the invention, the commercial Iowa Black® RQ quencher from IDT (Integrated DA Technologies) or “Quencher Black Hole” is used. Said quencher has an absorbance spectrum ranging from 500 to 700 nm with a peak absorbance at 656 nm and is ideal for use with fluorescent dyes emitting in the red spectral range.

### Construction of the Nano-Genosensor

The construction of the nano-genosensor was carried out following the methodology described by Huang, et al., 2013.

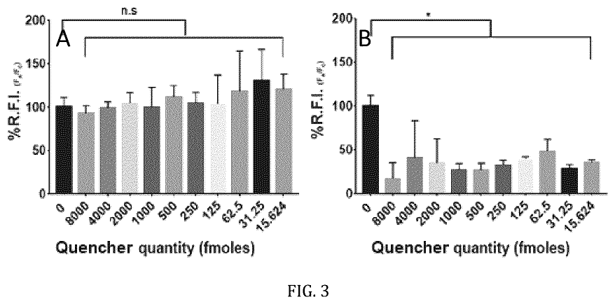

In order to test whether the functionalization of the nanoparticles (DNA probe binding on the surface of the nanoparticles) was successful, a comparison of percentages of fluorescence intensities between non-functionalized and functionalized CdTe-GSH QDs (FIGS. 3A and 3B, respectively) was performed by performing serial dilutions of QBH quencher-together with the functionalized and non-functionalized QDs separately, in a 1:2 volume ratio.

FIG. 3A shows that the QBH quencher does not absorb the fluorescence of the nanoparticle as the latter is not functionalized. The same experiment was performed, but this time with the nanoparticle functionalized with 4 nmoles of genomic probe. In FIG. 3B it is possible to appreciate the change generated by the nanoparticle when functionalized, and the decrease in the fluorescence emitted by these in the presence of different concentrations of QBH quencher.

### Types of Analyzed Samples

The nano-genosensor according to the invention can be used on samples of bovine saliva, milk, and serum (among others) prior DNA extraction, depending on the sample. In a preferred embodiment of the invention, the sample analyzed is selected from bovine saliva.

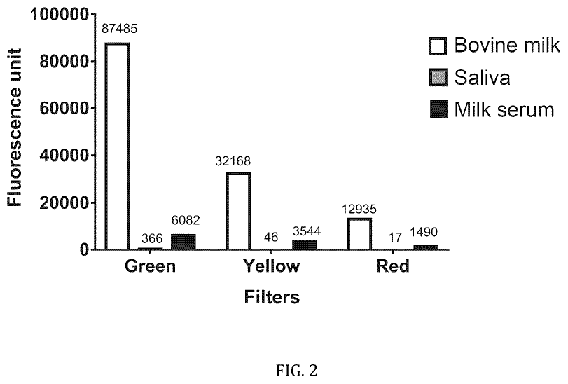

The working matrix was selected by comparing the autofluorescence of raw bovine milk, raw bovine milk serum and bovine saliva at different emission wavelengths of QDs (red 590 nm, yellow 540 nm, and green 460 nm) resulting in the lowest autofluorescence in bovine saliva in the emission of red nanoparticles (FIG. 2).

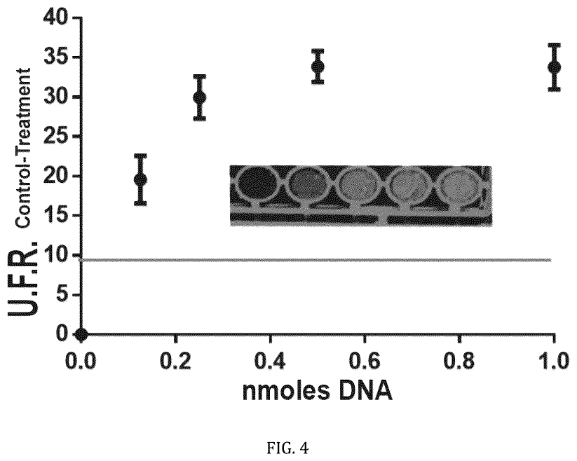

Accordingly, RD4 gene detection assays were performed using image processing to quantify the fluorescence present, together with the analysis of internal controls to discriminate the signal with statistical significance (FIG. 4).

### Methodology

It is possible to obtain an increase in fluorescence emission that is proportional to the amount of M. bovis DNA (RD4 gene) present in the samples.

The lower limit of detection is 0.1 nmoles (FIG. 4). Assays were standardized with 30 μg/mL of genomic-probe functionalized nanoparticle (ratio: 2 nmoles/mg of QDs) with M. bovis DNA (0, 0.96, 0.48, 0.24, 1.2 nmoles of DNA).

For the analysis of the results, the image of the microplate wells containing the nano-genosensor in the presence of M. bovis DNA is obtained, the image is processed to select the red spectrum (emission range) and analyzed on a computer using digital image processing software to generate a quantitative result in relative units of fluorescence intensity.

### Advantages of the Invention

Among the main advantages of the invention are the rapidity, sensitivity, specificity, and simplicity of use of the nano-genosensor. This allows the nano-genosensor to be used for on-site detection of bovine tuberculosis, as explained hereunder:
Matrix simplicity: considering that absorption and autofluorescence of biological matrices is a problem in obtaining a good quality optical signal, bovine saliva is an ideal matrix as it has lower autofluorescence compared to bovine milk and serum, which indicates that it allows following the fluorescence of QDs accurately. In addition, as in any respiratory transmitted disease, saliva is involved in the spread of the disease. Finally, bovine saliva does not discriminate in the analysis of M. bovis as to the gender of the animal, which unlike bovine milk or bovine milk serum as a working matrix would only allow analysis of lactating female cattle.
Speed: the proposed nano-genosensor allows obtaining instant results and shortening the time window to 2 hours versus the traditional tests that currently exist in the market, such as the tuberculin test or microbiological cultures that take 72 hours or more. In addition, the nano-genosensor, when applied in the field, allows testing to be done at any time and at any place, not depending on a professional for the application of the technology.
Analytical performance: Higher specificity/sensitivity (diagnostic accuracy) as it is based on direct molecular recognition of M. bovis DNA (hybridization with the probe) similar to PCR (it is a sort of “on-site PCR”) instead of an indirect immunological reaction (source of error with tuberculin).
Use of images: this allows the detection and quantification of fluorescence without the need for complex, expensive, and fragile equipment. This favors the use of this technology on site.

### EXAMPLES

### Example 1: Processing of Biosensor Nanoparticles

### 1.1 Synthesis of QDs

The synthesis of red CdTe-GSH QDs was performed based on the methodology of Pérez-Donoso (2012) using analytical grade materials (Sigma-Aldrich) which were: Glutathione (GSH), cadmium chloride (CdCl₂), potassium tellurite (K₂TeO₃), borate (Na₂B₄O₇×10H₂O) and citrate (C₆H₅Na₃O₇×2H₂O).

The reaction was carried out in 0.015 M borax-citrate buffer solution at pH 9.0, which was performed as follows:
measuring the pH of nanopure water in beaker;
adjusting pH to 9;
mixing 0.015 M borate with 0.015 M citrate in a 250 mL volumetric flask;
adding 200 mL of basic nanopure water (pH 9);
stirring until no residuals are visible;
transferring to a 500 mL laboratory vial;
adjusting pH to 9.5;
labelling and storing in refrigerator at 8° C.;

The synthesis reaction of red CdTe-GSH QDs was performed as follows:
adding 0.015 M GSH to a 15 mL centrifuge tube;
adding 0.015 M borax-citrate buffer at pH 9.5;
stirring in vortex;
adding CdCl₂ to a concentration of 0.1 M;
stirring vigorously in vortex;
adding K₂TeO₃ to a concentration of 0.1 M;
stirring quickly and vigorously to avoid the aggregation process;
aliquoting the solution into 1.6 mL microtubes;
incubating in thermoblock at 105° C. for 5 hours;
observing the tubes under a transilluminator at 330-360 nm;
collecting the solutions from the tubes in an amber vial;
the amounts of GSH, CdCl₂ and K₂TeO₃ used for the synthesis of 5 mL of quantum dots are: 0.023 g of GSH, 300 μL of CdCl₂ (0.1 M) and 50 μL of K₂TeO₃ (0.1 M).
   1.2 Functionalization with Thiolated Oligonucleotide

The functionalization of QDs with thiolated oligo was carried out using the methodology of Hill and Mirkin, 2006.
Reducing the disulfide group to thiol with 0.1 M Dithiothreitol (DTT) in cleavage buffer pH 8 in a 1.6 mL microtube.
Adding 5 nmol of 18 s (sense) oligonucleotide at a ratio of 5 nmol per 100 microliters of cleavage buffer.
Incubating for 2 hours in the dark at 22° C.

Purification of the reduced oligonucleotide was performed through a sephadex G-25 column (high cross-linked dextran, dry particle size: 20-50 uM previously hydrated).
Eluting the solution from the column until the meniscus touches the resin.
Adding the volume of oligonucleotide-DTT mixture.
Eluting until the sample enters into the column.
Discarding the eluted volume.
Adding 1 mL of nuclease-free nanopure water.
Eluting until the meniscus almost touches the resin.
Discarding the eluted volume.
Adding 1.5 times the volume of the nuclease-free nanopure water column.
Receiving volumes in 700 uL microtubes.
Quantifying the amount of reduced oligonucleotide through a Take 3 plate in absorbance equipment at a wavelength of 260-280 nm.
Mixing CdTe-GSH Red QDs with the thiolated oligonucleotide at a ratio of 1 mg/mL QDs for 2, 4 and 6 nmoles of thiolated oligonucleotide.
Incubating at 22° C. for 2 days.

The functionalization of the CdTe-GSH QDs with the thiolated oligonucleotide was tested by performing two experiments.
i) Mixing functionalized nanoparticles with QBH quencher.
ii) Mixing non-functionalized nanoparticles with QBH quencher.

### 1.3 Quencher

Commercial Quencher Black Hole (QBH) from Itegrated DNA Technologies (IDT) was used with the sequence complementary to the thiolated oligonucleotide sequence already bond to it.

The QBH probe was reconstituted with nuclease-free nanopure water and remained at a stock concentration of 20 uM for further work.

### Example 2: Results of Use of the Biosensing Nanoparticles According to the Invention

### Construction of the Nano-Genosensor

Validation assays for detection of M. bovis genomic DNA (provided by SAG) were performed using 1 mg of QDs and different amounts of the genomic probe (2, 4 and 6 nmoles) and quencher bond to the nanoparticle (once and twice in relation to the DNA probe).
Mixing 100 μL of functionalized QDs (1 mg/mL) with 50 μL of QBH quencher (twice as high).
Adding 100 μL of M. bovis DNA (RD4 gene).
Incubating at 95° C. for 10 minutes.
Transferring volumes to 96-well microplate.
Displaying through transilluminator.

### Example 3: Comparative Results in Relation to Similar Technologies

The nano-genosensor surpasses the tuberculin test (gold standard in tuberculosis detection) in the following parameters:
(i) Speed: it allows results in 2 h, thus shortening the response time of the tuberculin test (72 h);
(ii) Analytical performance: increased specificity/sensitivity (diagnostic accuracy) by relying on direct molecular recognition of M. bovis DNA (hybridization with the probe);
(iii) Digital device: automation and objectivity associated with quantitative response, not depending on interpretation of skin lesions;
(iv) Simplicity of the matrix: the use of saliva simplifies sampling and pretreatment, in addition to not discriminating animal gender.

Table 1 shows a comparative table summarizing the main features in relation to available screening and confirmatory methods (PCR and bacterial culture). The nano-genosensor offers the speed/portability features of screening tests but combined with the high analytical performance of molecular confirmatory methods.

TABLE 1
Comparative table of features of the nano-genosensor (technological solution
of the present invention) and commercially available methods for detection
of M. bovis in cattle including screening and confirmatory methods

| | | Portability | Speed of | Analytical | | Laboratory |
|---|---|---|---|---|---|---|
| | Type of | (on-site | the test | performance: | Digital | qualified |
| METHOD | Sample | use) | (on-line) | (sensitivity/specificity) | device | professional |
| Tuberculin | In vivo | ✓ | X | X | X | ✓ |
| test (TB only) | | | | | | |
| ELISA | Milk - | X | ✓ | ✓ | ✓ | ✓ |
| (Bovigam Kit) | Serum | | | | | |
| Molecular | Milk - | X | X | ✓ | ✓ | ✓ |
| methodologies | Serum - | | | | | |
| (RT-PCR, | Animal | | | | | |
| Boviman kit) | tissues | | | | | |
| Bacteriological | Milk - | X | X | ✓ | X | ✓ |
| culture (gold | Serum - | | | | | |
| standard) | Animal | | | | | |
| | tissues | | | | | |
| Nano- | Milk - | ✓ | ✓ | ✓ | ✓ | X |
| genosensor | Saliva | | | | | |
| device | | | | | | |
| (solution kit) | | | | | | |

1. A nano-genosensor for detecting the presence of Mycobacterium bovis, CHARACTERIZED in that it comprises:
a. fluorescent semiconductor nanoparticles;
b. genomic probes; and
c. a fluorescence quencher coupled with the sequence complementary to the genomic probe sequence;
wherein the nanoparticles are functionalized with the genomic probe.

2. A nano-genosensor according to claim 1, CHARACTERIZED in that the fluorescent semiconductor nanoparticles consist of Cadmium Telluride.

3. A nano-genosensor according to claim 2, CHARACTERIZED in that the nanoparticles are made from Glutathione (GSH), cadmium chloride (CdCl₂) and potassium tellurite (K₂TeO₃).

4. A nano-genosensor according to claim 3, CHARACTERIZED in that the glutathione, cadmium chloride and potassium telluride are in a molar ratio of 10-15:4-6:1.

5. A nano-genosensor according to claim 1, CHARACTERIZED in that the ratio of genomic probe and nanoparticles is 2 to 6 nmoles/mg of nanoparticles.

6. A nano-genosensor according to claim 1, CHARACTERIZED in that the genomic probe is a sequence complementary to the RD4 gene of M. bovis corresponding to SEQ ID sequence No. 1.

7. A nano-genosensor according to claim 1, CHARACTERIZED in that the quencher is coupled with the sequence complementary to SEQ ID sequence No. 1.

8. A nano-genosensor according to claim 7, CHARACTERIZED in that the fluorescence quencher preferably corresponds to a quencher in an absorbance spectrum ranging from 500 to 700 nm.

9. A method for detecting the presence of Mycobacterium bovis, CHARACTERIZED in that it comprises contacting a sample of bovine saliva, milk, or serum with the nano-genosensor according to any one of claim 1 and performing detection from the fluorescence intensity.

10. A method according to claim 9, CHARACTERIZED in that the sample preferably corresponds to bovine saliva.
